(19) United States
(12) Patent Application Publication
(10) Pub. No.: US 2010/0317738 A1
(43) Pub. Date: Dec. 16, 2010

(54) COMPOSITIONS AND METHODS FOR TREATING CONDITIONS ASSOCIATED WITH NEURONAL DYSFUNCTION

(75) Inventors: Marco Martina, Chicago, IL (US); A. Vania Apkarian, Chicago, IL (US)

Correspondence Address: Towsend and Towsend and Crew LLP, Two Embarcadero Center, 8th Floor, San Francisco, CA, 94111, US

(73) Assignee: NORTHWESTERN UNIVERSITY, Evanston, IL (US)

(21) Appl. No.: 12/781,282

(22) Filed: May 17, 2010

Related U.S. Application Data
(60) Provisional application No. 61/178,798, filed on May 15, 2009.

(51) Int. Cl. A61K 31/196; A61P 25/00

(52) U.S. Cl. 514/567

(57) ABSTRACT

The present invention relates to compositions and methods for the treating and empirically investigating conditions associated with neuronal dysfunction (e.g., chronic pain, epileptic neuronal activity). In particular, the present invention provides compositions and methods for using flufenamic acid in the treatment and empirical investigation of conditions associated with neuronal dysfunction (e.g., chronic pain, epileptic neuronal activity).

The present application claims priority to U.S. Provisional Patent Application Ser. No. 61/178,798, filed May 15, 2009, the entire disclosure of which is herein incorporated by reference in its entirety.

This invention was made with government support under 5R01NS042660 awarded by the National Institute of Health. The government has certain rights in the invention.

### FIELD OF THE INVENTION

The present invention relates to compositions and methods for the treating and empirically investigating conditions associated with neuronal dysfunction (e.g., chronic'pain, epileptic neuronal activity). In particular, the present invention provides compositions and methods for using flufenamic acid in the treatment and empirical investigation of conditions associated with neuronal dysfunction (e.g., chronic pain, epileptic neuronal activity).

### BACKGROUND OF THE INVENTION

Chronic pain is defined as pain that persists longer than the temporal course of natural healing, associated with a particular type of injury or disease process (see, e.g., Shipton EA, et. al., (2005) European journal of anaesthesiology 22 (6): 405-12; herein incorporated by reference in its entirety). The International Association for the Study of Pain defines pain as an unpleasant sensory and emotional experience associated with actual or potential tissue damage, or described in terms of such damage (see, e.g., Merskey H (1994). “Logic, truth and language in concepts of pain”. Quality of life research: an international journal of quality of life aspects of treatment, care and rehabilitation 3 Suppl 1: S69-76; herein incorporated by reference in its entirety). It is important to note that pain is subjective in nature and is defined by the person experiencing it, and the medical community's understanding of chronic pain now includes the impact that the mind has in processing and interpreting pain signals.

Many primary conditions, whether acute (e.g. injury), recurring (e.g. migraine), or chronic (e.g. arthritis) are significantly complicated by co-morbid pain disorders. Some pain conditions are unassociated with other primary diagnoses. Chronic pain is widely considered a disease itself, causing long-term detrimental physiologic changes and requiring unique assessments and treatments. While acute pain is a normal sensation triggered in the nervous system to alert a subject to possible injury, chronic pain is different. Chronic pain persists. Pain signals keep firing in the nervous system for days, weeks, months, even years. Chronic pain may be instigated by an initial event (e.g. sprained back, serious infection), or there may be an ongoing cause of pain (e.g. arthritis, cancer, ear infection), but some people suffer chronic pain in the absence of any past injury or evidence of body damage. Many chronic pain conditions affect older adults. Common chronic pain complaints include headache, low back pain, cancer pain, arthritis pain, neurogenic pain (e.g. pain resulting from damage to the peripheral nerves or to the central nervous system itself), psychogenic pain (e.g. pain not due to past disease or injury or any visible sign of damage inside or outside the nervous system).

Complex regional pain syndrome (CRPS) is a chronic pain condition. The key symptom of CRPS is continuous, intense pain out of proportion to the severity of the injury, which gets worse rather than better over time. CRPS most often affects one of the arms, legs, hands, or feet. Typical features include dramatic changes in the color and temperature of the skin over the affected limb or body part, accompanied by intense burning pain, skin sensitivity, sweating, and swelling. The cause(s) of CRPS are unknown. In some cases, the sympathetic nervous system plays an important role in sustaining the pain. CRPS may be caused by a triggering of the immune response, which leads to the characteristic inflammatory symptoms of redness, warmth, and swelling in the affected area. Because there is no cure for CRPS, treatment is aimed at relieving painful symptoms. Doctors may prescribe topical analgesics, antidepressants, corticosteroids, and opioids to relieve pain. However, no single drug or combination of drugs has produced consistent long-lasting improvement in symptoms. Other treatments may include physical therapy, sympathetic nerve block, spinal cord stimulation, and intrathecal drug pumps to deliver opioids and local anesthetic agents via the spinal cord.

Improved methods for treating and empirically investigating pain (e.g. chronic pain, non-inflammatory pain, CRPS, etc.) are needed.

### SUMMARY OF THE INVENTION

Experiments conducted during the course of development of embodiments for the present invention demonstrated that flufenamic acid (FFA) to be effective in the treatment of conditions associated with neuronal dysfunction (e.g., chronic pain, neuronal epileptic activity, CRPS, etc.). Accordingly, the present invention provides pharmaceutical compositions and methods for treating and/or preventing such conditions.

In certain embodiments, the present invention provides methods for treating and/or preventing a condition associated with neuronal dysfunction. In some embodiments, the methods comprise administering to a subject suffering from neuronal dysfunction a pharmaceutical composition comprising flufenamic acid. In some embodiments, the subject is a human being. In some embodiments, the subject is, for example, a mouse, a cat, a rat, a gorilla, a cow, a sheep, and/or a dog. The methods are not limited to a particular condition associated with neuronal dysfunction. In some embodiments, the condition associated with neuronal dysfunction is chronic pain. In some embodiments, the condition associated with neuronal dysfunction is epileptic activity. In some embodiments, the pharmaceutical composition is co-administered with one or more therapeutic agents. The methods are not limited to particular therapeutic agents. Examples of therapeutic agents include, but are not limited to, antidepressants, selective serotonin reuptake inhibitors, opioids, anticonvulsants, analgesics, nonsteroidal anti-inflammatory drugs, carbamazepine, clobazam, clonazepam, ethosuximide, felbamate, fosphenyloin, flurazepam, gabapentin, lamotrigine, levetiracetam, oxcarbazepine, mephenyloin, phenobarbital, phenyloin, pregabalin, primidone, sodium valproate, tiagabine, topiramate, valproate semisodium, valproic acid, vigabatrin, diazepam, lorazepam, paraldehyde, pentobarbital, and bromides.

The methods are not limited to a particular manner for treating and/or preventing a condition associated with neuronal dysfunction through administration of a pharmaceutical composition comprising FFA. In some embodiments, the methods involve activation of KCNK potassium channels. In some embodiments, the methods involve activation of activation of neuronal background potassium conductance. In some embodiments, the methods involve activation of inhibition of neuronal voltage-gated sodium current.

In some embodiments, the treatment is used to prevent or alleviate chronic pain. In some embodiments, the chronic pain is not caused by chronic inflammation and/or is not associated with a chronic inflammatory disease or condition. Thus, in some embodiments, the subject treated does not suffer from a disease or condition associated with chronic inflammation (e.g., rheumatoid arthritis). In some embodiments, the subject does not suffer from osteoarthritis, inflammatory arthropathies (e.g., ankylosing spondylitis, psoriatic arthritis, Reiter's syndrome), acute gout, dysmenorrhoea, metastatic bone pain, headache, migraine, post operative pain, tissue injury, tissue inflammation, pyrexia, ileus, or renal colic. However, in some embodiments, subjects do suffer from one or more of the above diseases or conditions, but experience chronic pain that is not addressable via traditional NSAIDs such as aspirin, ibuprofen, and naproxen or other agents whose anti-pain activity functions through the inhibition of cyclooxygenase enzymes (e.g., COX-1 and/or COX-2).

In some embodiments, the subject suffers from pain that is treatable by slowing the onset of neuronal Na⁺ channel inactivation and/or reducing burst firing of such neurons. Thus, in some embodiments, the present invention provides for the use of FFA or related compounds for the treatment of pain that is treatable by slowing the onset of neuronal Na⁺ channel inactivation or burst firing (e.g., in hippocampal neurons). In some embodiments, the pain to be treated is experience at a distal location in the body from the site of action of the drug (e.g., the pain is felt by the subject outside of the head, outside of the brain, etc.).

In some embodiments, the present invention provides the use of FFA or a pharmaceutically acceptable salt thereof in the manufacture of a therapeutic to prevent or alleviate chronic pain. In some embodiments, the pain comprises chronic pain. In some embodiments, the pain comprises idiopathic pain. In some embodiments, the pain comprises CRPS. In some embodiments, the therapeutic comprises an effective amount of a second therapeutic agent. In some embodiments, the second therapeutic targets pain. In some embodiments, the second therapeutic targets an underlying cause or issue (e.g. disease, disorder, or condition) secondary to the pain. In some embodiments, the present invention provides the use of FFA or a pharmaceutically acceptable salt thereof in the manufacture of a therapeutic for slowing the onset of neuronal Na⁺ channel inactivation and/or reducing burst firing of such neurons.

### BRIEF DESCRIPTION OF THE DRAWINGS

FIG. 1 shows that flufenamic acid has potent analgesic effect in rats. Von Frey thresholds (a standard measure of pain sensitivity) were measured on the operated paw (left panel) and on the contralateral paw (right panel) in adult rats. 4 animals received the drug (10 mg/kg oral administration), and 4 animals the vehicle only. As shown in the left panel, flufenamic acid increased pain threshold in both paws, with a dramatic effect on the injured paw (the threshold increased more than 10 times). Note the fast onset of the effect, which may be very important for clinical use.

FIG. 2 shows TRAAF-like currents in DRG neurons. Patch clamp measurements of the currents (middle traces) elicited in a rat DRG neuron by a slow depolariring ramp (upper trace) in control condition and in the presence of the TRAAK blocker gadolinium (20 pM, red trace). Lower trace: gadolinium-sensitive current obtained by digital subtraction.

FIG. 3 shows DRG expression of TRAAK mRNA is altered in neuropathic pain. Expression of TRAAK transcript in contralateral and ipsilateral lumbar DRGs was examined by real time PCR in an SNI rat. Data are normalized to transcript of β-actin to eliminate potential differences in the total amount of tissue. This data suggest, for example, that the effect of flufenamic acid on these channels may be selective for injured neurons.

FIG. 4 shows flufenamic acid abolishes epileptic discharges in an in vitro model of epilepsy. A, extracellular recordings from the CA1 area of a rat hippocampal slices in which epileptic-like activity was induced by bath application of 4AP (50 μM). The lower trace shows the indicated area at a higher time resolution. Flufenamic acid (FFA, 200 μM) completely and reversibly inhibited the epileptiform discharges. B, in another 4AP treated slice, the effects of different concentrations of FFA were tested. FFA showed a clear dose-dependent effect. At 50 μM, FFA main effect was a small reduction in the amplitude of individual events. At 100 μM, FFA strongly reduced the discharge frequency. At 200 μM, FFA completely abolished the discharges.

FIG. 5 shows voltage dependence of sodium channel inactivation in CA1 pyramidal neurons. IN plots obtained from the averaged values of 2 nucleated patch recordings in control conditions (full symbols) and in the presence of FFA (empty symbols). Holding potential was −90 mV. 100 ms pre-pulses to the voltages indicated in the plot preceded a 30 ms test pulse to 0 mV. Data were fit with Boltzmann functions (solid lines). Fit values for the control condition were: function half point: −65.9 mV; slope 11.4 mV. In the presence of FFA the values were: function half point: −89.7 mV; slope: 9.1.

FIG. 6 shows a computational model of the flufenamic acid effect on sodium current gating. (A) Schematics of model states and transitions between them. The upper row corresponds to the slow inactivated states while the bottom row corresponds to the fast inactivated states. The middle row includes two closed states and one open state. Only the open state passes current. The forward transition rates are shown above/on left while the backward rates are shown below/on right of the corresponding transition arrows. (B) The time constants of inactivation processes calculated as τ=(+β)⁻¹. The slow inactivation rates were unaffected by FFA. (C) Currents simulated with a model during the voltage clamp protocol shown on the top (lighter wide traces) are overlaid with traces obtained during recordings from hippocampal neurons (dark thin traces). (D) Simulated currents evoked during voltage clamp protocol shown on the top. The black trace was obtained with a control model while the red trace represents the FFA modified model. (E/F), Current clamp traces simulated with control (E) and FFA modified (F) model. Scale bars correspond to 2 ms and 100 pA in C and to 200 ms and 20 mV in E and F.

FIG. 7 shows flufenamic acid reduces voltage-gated sodium current in hippocampal pyramidal neurons. (A) Na⁺ currents recorded from one nucleated patch first in control condition (left) and then in the presence of 200 μM FFA (right); a 50 ms prepulse to −70 mV preceded the 30 ms test pulse to −10 mV (bottom); patches were held at −90 mV to avoid slow inactivation. (B) Bar chart showing of the mean peak Na⁺ current amplitude in control and in FFA in 15 nucleated patches. FFA reduced the current from −277.9±26.3 pA in control to −183.6±21.7 pA.

FIG. 8 shows flufenamic acid has minimal effect on Na⁺ channel activation. (A)

Representative traces of Na⁺ currents recorded from nucleated patches in control conditions (top traces) and in the presence of 200 μM FFA (middle traces). Bottom traces show the stimulation pulse protocol; the patch was stepped to −120 mV for 50 ms, then depolarized by a family of 30 ms-long pulses between −70 mV and +40 mV (10 mV steps), and back to −90 mV; holding potential was −90 mV. (B) Na permeability in control conditions (hollow symbols) and in the presence of FFA (solid symbols) normalized to the maximum value and plotted against test pulse potential. The experimental data were fitted with Boltzmann curves raised to third power. No significant difference was found in mid-point potential between control (−12.5±1.8 mV) and FFA treatment (−11.3±1.36 mV) while the value of the slope factor (k/3) in FFA (5.6±0.27 mV) was slightly larger than in control.

FIG. 9 shows flufenamic acid slows the onset of Na⁺ channel inactivation. (A) The time course of onset of Na channels inactivation at 10 mV (30 ms pulse) in control and in the presence of FFA. Na⁺ currents were recorded using test pulses between −30 mV and 40 mV in 10 mV increments following a 50 ms-long pre-pulse to −120 mV. The traces shown were normalized to their respective peak current amplitude and superimposed to better illustrate the effect of FFA on the current inactivation and fit using single exponential curves (dashed lines). (B) Na⁺ current inactivation time constant at different potentials in control (hollow symbols) and in FFA (solid symbols). The time course of inactivation onset could be fit with a single exponential function at all membrane potentials tested.

FIG. 10 shows flufenamic acid shifts Na⁺ current steady-state inactivation and decreases Na⁺ availability at resting membrane potential. (A/C) Na⁺ currents recorded in control and in the presence of FFA. Nucleated patches were held at −90 mV, and currents were elicited by 30 ms test pulses to −10 mV preceded by either 50 ms (A) or 200 ms (C) prepulses (from −120 to −30 mV, 10 mV steps, insets). (B/D) Steady state inactivation curves with 50 ms (B) or 200 ms (D) prepulses in control (hollow symbols) and in FFA (solid symbols). Peak Na⁺ current amplitudes were normalized to the maximum current, plotted against prepulse potential value, and fit by simple Boltzmann equations. For 50 ms prepulse, the mid-point potential and slope factor were −55.4±1.3 mV and 8.3±0.4 mV in control, and −67.8±1.4 mV and 10.7±0.5 mV in FFA. For 200 ms prepulse, the mid-point potential and slope factor were −64.1±2.2 mV and 7.5±0.2 mV in control, and −74.1±2.6 mV and 8.9±1.0 mV in FFA, respectively.

FIG. 11 shows flufenamic acid slows the recovery of Na⁺ channel from inactivation. Recovery from fast inactivation of Na⁺ currents in control (A) and in the presence of FFA (B). A double-pulse protocol was used to measure the extent of recovery from inactivation. Patches were held at −90 mV, and Na⁺ currents were elicited by a 30 ms test pulse to −10 mV (following a 50 ms prepulse to −120 mV); a second identical test pulse at −10 mV was then applied after increasing time intervals at −120 mV. These traces were obtained using interpulse intervals from 1 to 12 ms and normalized to the peak current elicited by the respective first pulses for better comparison. (C) Time-course of recovery from inactivation in control and in FFA. The amplitude of Na⁺ current evoked by the second test pulse was normalized to that of the first test pulse and plotted against the interpulse interval. Data points were fit using double exponential functions. The fast and slow components of recovery time constants were 2.09 ms and 84.45 ms in control, and 7.72 and 61.32 ms in FFA.

FIG. 12 shows comparison of the inactivation properties of simulated and experimentally measured currents. Experimental data points are represented by continuous fit functions while filled circles represent simulated current measurements.

FIG. 13 shows flufenamic acid decreases pyramidal cell firing upon depolarizing current injection. (A) Current clamp recordings obtained (at 32-33° C.) from a CA1 hippocampal neuron in response to depolarizing current injection (150 pA, 1 s, bottom) in control (top trace) and in the presence of FFA (200 μM, middle). (B) Firing frequency in response to a series of current injections (1 s, 30 pA steps) in control and in FFA.

FIG. 14 shows flufenamic acid decreases 4-AP induced bursting in CA1 pyramidal neurons. (A) Current clamp recordings obtained from a CA1 hippocampal neuron in response to depolarizing current (120 pA, 1 s) injection in the presence of 4-AP (100 μM, left panel) and 4AP+FFA (100 μM, right panel). The trace segments between dashed lines are shown on an expanded time scale (insets) to resolve bursts. (B) Firing frequency in response to depolarizing current injections. (C) Interspike interval ratio (ISI) against different current injections. For each current step, the ISI ratio was obtained dividing the interval between the first two spikes by the mean interspike interval. Recordings were obtained at 24-25° C.

### DETAILED DESCRIPTION OF THE INVENTION

Flufenamic acid (2-[[3-(trifluoromethyl)phenyl]amino]benzoic acid) (FFA) (MERALEN) is a non-steroidal anti-inflammatory drug (see, e.g., Seligra A, et al., Curr Med Res Opin. 1990; 12(4):249-54; Dawood MY Drugs 1981 July; 22(1):42-56; Kagan G, et al., J Int Med Res 1981; 9(4):253-6; each herein incorporated by reference in their entireties). In experiments conducted during the course of development of embodiments for the present invention, FFA was shown to be effective in the treatment of conditions associated with neuronal dysfunction. Accordingly, the present invention provides pharmaceutical compositions and methods for treating and/or preventing conditions associated with neuronal dysfunction. The present invention is not limited to treatment and prevention of neuronal dysfunction or any type of neuronal dysfunction. In some embodiments, the present invention provides compositions and methods for treatment, prevention, and/or relief of symptoms of pain, chronic pain, non-inflammatory pain, etc. In some embodiments, the present invention provides compositions for co-administration with other therapies for the treatment, prevention, and/or symptom relief of pain (e.g. caused by neuronal dysfunction; caused by an underlying disorder, disease, or condition; of unknown cause, non-inflammatory pain, inflammatory pain, chronic pain, etc.), and method of co-administration. In some embodiments, the present invention provides administration of FFA for treatment, prevention, and/or symptom relief of pain (e.g. caused by neuronal dysfunction; caused by an underlying disorder, disease, or condition; of unknown cause, non-inflammatory pain, inflammatory pain, chronic pain, CRPS, etc.). In some embodiments, the present invention provides co-administration of FFA and one or more additional therapies (e.g. pharmaceuticals, physical therapy, psychiatry, etc.) for treatment, prevention, and/or symptom relief of pain (e.g. caused by neuronal dysfunction; caused by an underlying disorder, disease, or condition; of unknown cause, non-inflammatory pain, inflammatory pain, chronic pain, etc.).

In some embodiments, the present invention provides compositions (e.g. FFA) and methods (e.g. co-therapy (e.g. co-administration)) for the treatment and/or prevention of pain. In some embodiments, the present invention is not limited to any type of pain. In some embodiments, the present invention provides treatment and/or prevention of: inflammatory pain, non-inflammatory pain, pain secondary to an underlying disorder, pain of unknown origin, visceral pain, chronic urologic pelvic pain syndromes, neuropathic pain, spinal cord injury pain, headache pain, musculoskeletal pain (e.g. back pain), cancer pain, bone pain secondary to cancer, cardiovascular pain disorders, pain from chemotherapy-induced neuropathies and other malignant disorders, pain from fibromyalgia, pain from temporomandibular joint and muscle disorders, pain associated with HIV/AIDS, pain associated with osteoporosis, pain associated with communication disorders (e.g., otitis media, tinnitus, burning mouth syndrome, dysphagia), end of life pain, pain in older persons with multiple contributing morbidities, pain in people with drug and alcohol addictions, pain in persons with neuromuscular conditions, pain in preterm neonates exposed to multiple medical interventions, pain resulting from skin disorders, etc. In some embodiments, the treated pain is idiopathic pain, nociceptive pain, neuropathic pain, or psychogenic pain.

In some embodiments, compositions of the present invention (e.g. FFA, derivatives of FFA, modified FFA, etc.) are co-administered with one or more additional therapeutics. In some embodiments, a therapeutic co-administered with a composition of the present invention (e.g. FFA) treats and/or prevents an underlying cause of pain. In some embodiments, a therapeutic co-administered with a composition of the present invention (e.g. FFA) targets a different pathway than the composition of the present invention (e.g. FFA). In some embodiments, a therapeutic co-administered with a composition of the present invention (e.g. FFA) treats and/or prevents a condition, disease, and/or disorder associated with, secondary to, or peripheral to, pain (e.g. chronic pain, CRPS, etc.). In some embodiments, a therapeutic co-administered with a composition of the present invention (e.g. FFA) comprises a pain-relief therapeutic, including, but not limited to: non-narcotic analgesics (e.g. acetaminophen), non-steroidal anti-inflammatory drugs (NSAIDs; e.g. diclofenac, diflunisal, etodolac, fenoprofen, flurbiprofen, ibuprofen, indomethacin, ketoprofen, ketorolac, meclofenamate, mefenamic acid, meloxicam, nabumetone, naproxen, oxaprozin, phenylbutazone, piroxicam, sulindac, tolmetin, etc.) COX-2 inhibitors (e.g. celecoxib, etc.), central analgesics (e.g. tramadol, etc.), narcotic pain medications (e.g. buprenorphine, butorphanol, codeine, hydrocodone, hydromorphone, levorphanol, meperidine, methadone, morphine, nalbuphine, oxycodone, oxymorphone, pentazocine, propoxyphene, etc.), topical analgesics (e.g. capsaicin, etc.), topical anesthetics (e.g. benzocaine, dibucaine, lidocaine, prilocalne, etc.), and combinations thereof. In some embodiments, FFA is co-administered with one or more pharmaceuticals including, but not limited to: carbamazepine, clobazam, clonazepam, ethosuximide, felbamate, fosphenyloin, flurazepam, gabapentin, lamotrigine, levetiracetam, oxcarbazepine, mephenyloin, phenobarbital, phenyloin, pregabalin, primidone, sodium valproate, tiagabine, topiramate, valproate semisodium, valproic acid, vigabatrin, diazepam, lorazepam, paraldehyde, pentobarbital, and bromides.

The methods are not limited to a particular method for treating and/or preventing a condition associated with neuronal dysfunction and/or pain (e.g. chronic pain, CRPS, non-inflammatory pain, etc.). In some embodiments, the method for treating and/or preventing a condition associated with neuronal dysfunction and/or pain (e.g. chronic pain, CRPS, non-inflammatory pain, etc.) comprises administering to a subject a pharmaceutical composition comprising FFA or functional equivalent derivatives thereof. In some embodiments, the method for treating and/or preventing a condition associated with neuronal dysfunction and/or pain (e.g. chronic pain, non-inflammatory pain, etc.) comprises administering to a subject a pharmaceutical composition comprising FFA with one or more additional therapeutic agents (e.g., a therapeutic agent capable of treating and/or preventing a condition associated with neuronal dysfunction and/or pain (e.g. chronic pain, non-inflammatory pain, etc.)). The methods are not limited to a particular type of subject (e.g., a cat, a dog, a rodent, a primate). In some embodiments, the subject is a human being. The methods are not limited to a particular conditions associated with neuronal dysfunction (e.g., chronic pain, CRPS, epileptic neuronal activity). In some embodiments, the condition associated with neuronal dysfunction is chronic pain. In some embodiments, the condition associated with neuronal dysfunction is non-inflammatory pain. In some embodiments, the condition associated with neuronal dysfunction is epilepsy. In some embodiments, the condition associated with neuronal dysfunction is CRPS. In some embodiments, the condition associated with neuronal dysfunction involves dysfunctional neuronal background potassium conductance. In some embodiments, the condition associated with neuronal dysfunction involves dysfunctional neuronal voltage-gated sodium current. In some embodiments, the condition associated with neuronal dysfunction involves dysfunctional neuronal KCNK potassium channel activity.

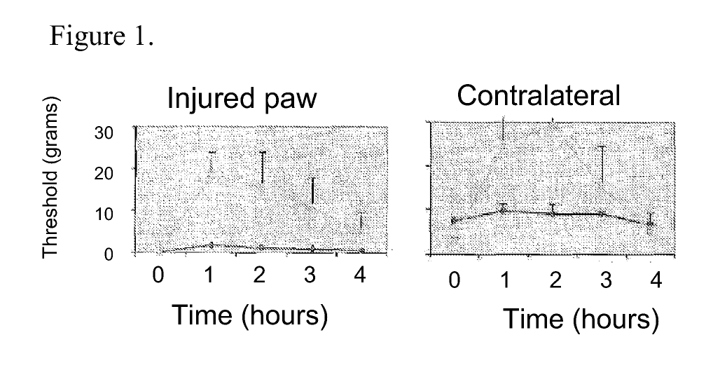

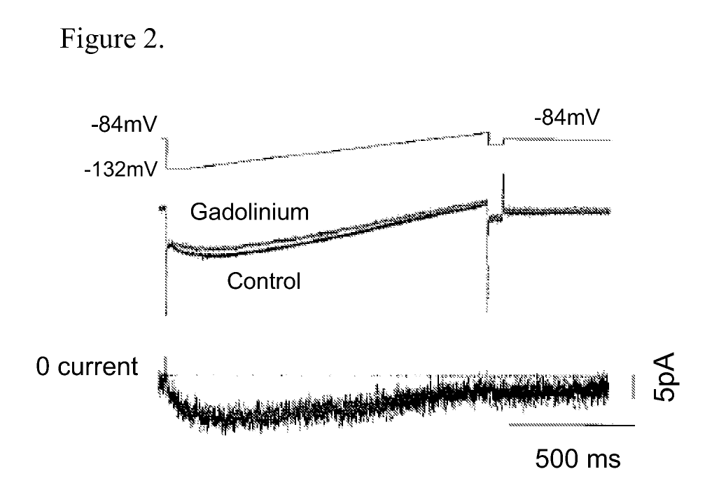

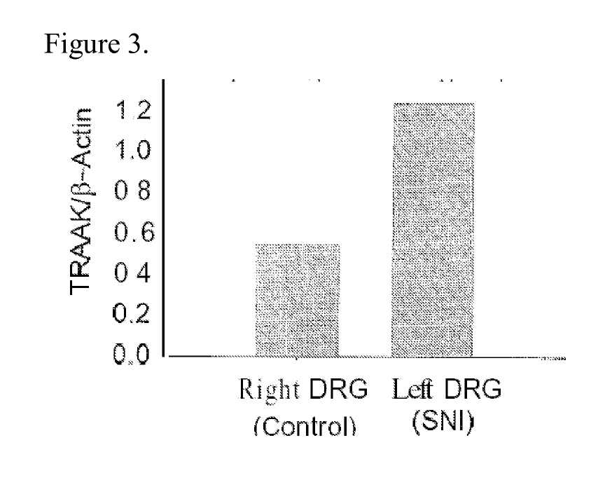

Chronic pain constitutes a primary reason driving people to seek health care. Pain was has been described as “epidemic” by the American Academy of Pain Management (American Pain Society, 1999): 50 million Americans are partially or totally disabled by pain. Experiments conducted during the development of embodiments for the present invention demonstrated that FFA has a potent analgesic effect in an animal model of neuropathic pain. In particular, the spared nerve injury model (SNI), a reliable animal model of chronic neuropathic pain, was used to test the effect of oral administration of FFA. Control experiments were performed administrating the vehicle only. FIG. 1 shows that FFA (10 mg/kg, in ethanol) had a rapid and potent analgesic effect in SNI rats. FFA also significantly increased the pain threshold in the non-operative paw. This observation demonstrated that the FFA effect is not attributable to the NSAID effect of flufenamic acid. FFA has been shown to activate KCNK potassium channels (e.g., currents mediated by channels containing TREK and TRAKK subunits) (see, e.g., Takahira M, et al., 2005, Pflugers Arch December; 451(3):474-8 Epub 2005 August 2; herein incorporated by reference in its entirety). KCNK channels regulate neuronal excitability by regulating membrane potential, input resistance and synaptic gain (see, e.g., Taverna S, et al., 2005, J Neurosci October 5; 25(40):9162-70; Meuth S G, et al., 2003 J. Neurosci. July 23; 23(16):6460-9; each herein incorporated by reference in their entireties). Although no highly selective blockers of KCNK currents exist, gadolinium can be used to block currents mediated by TRAKK subunits (see, e.g., Maingret F, et al., J Biol Chem 1999 Jan. 15; 274(3):1381-7; herein incorporated by reference in its entirety). FIG. 2 shows that a gadolinium-sensitive potassium current (that is, a TRAKK-mediated current) can be recorded from DRG these neurons. FIG. 3 shows that expression of TRAKK channel protein (mediating gadolinium-sensitive background channels) is increased in animals with neuropathic pain offering an ideal and selective target for FA. In a set of parallel experiments, performed on hippocampal neurons, FFA was shown to be a gating modifier of voltage.

In some embodiments, the present invention provides compositions and methods for treating and/or preventing chronic pain comprising administering to a subject a pharmaceutical composition comprising FFA. The methods are not limited to a particular mechanism for treating and/or preventing chronic pain in a subject. In some embodiments, the administered FFA treats and/or prevents chronic pain through activation of KCNK potassium channels. The methods are not limited to administering a particular amount of FFA. In some embodiments, the amount of administered FFA is sufficient to activate KCNK potassium channels. In some embodiments, the amount of administered FFA is sufficient to increase the pain threshold for the subject. In some embodiments, additional therapeutic agents are co-administered with the compositions comprising FFA. The methods are not limited to particular additional therapeutic agents. In some embodiments, the additional therapeutic agents include, but are not limited to, antidepressants (e.g., amitriptyline, nortriptyline), selective serotonin reuptake inhibitors (e.g., fluoxetine, sertraline, paroxetine), opioids (e.g., oxycodone, fentanyl), anticonvulsants (e.g., gabapentin), analgesics (e.g., acetaminophen), and nonsteroidal anti-inflammatory drugs (e.g., ibuprofen, naproxen sodium).

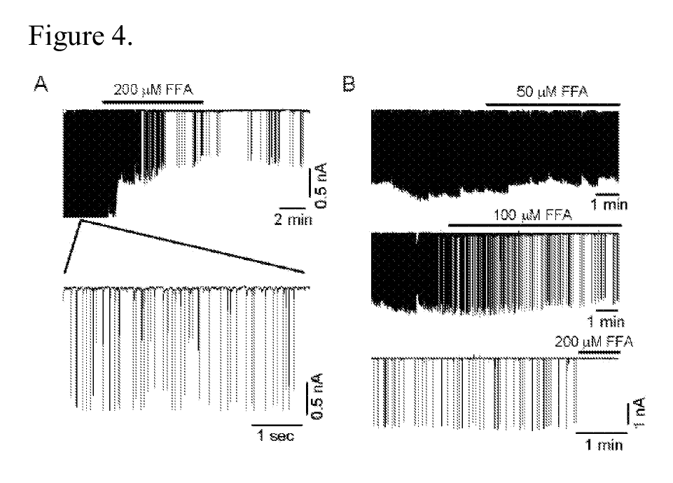

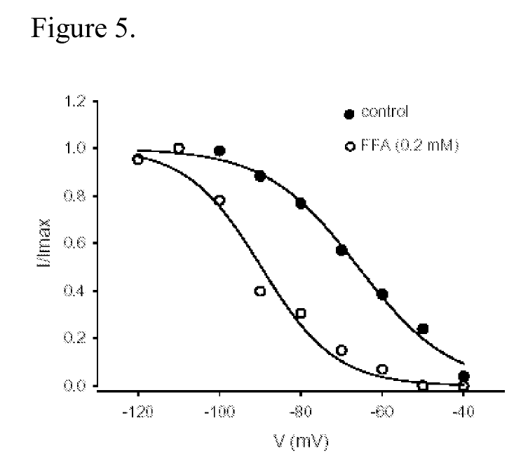

Similar pathophysiologies exist for epilepsy and various chronic pain disorders (see, e.g., Pappagallo M (2003) Clin Ther. 25:2506-38; herein incorporated by reference in its entirety). Experiments conducted during the development of embodiments for the present invention demonstrate that FFA is an effective antiepileptic drug in a model of epilepsy. Epileptic discharges were induced in a slice of rodent hippocampal tissue by bath application of the voltage-gated potassium channel blocker 4-aminopyridine (4AP) (see, e.g., Lopantsev V, Avoli M. (1998) J. Physiol. 509:785-96; Bruckner C, et al., Neurosci Lett. 268:163-5; each herein incorporated by reference in their entireties). FIG. 4 shows that FFA has a potent, concentration dependent, antiepileptic effect in this epilepsy model. The effect of flufenamic acid on hippocampal neurons, for example, involves induction of neuronal hyperpolarization by activating a background potassium conductance, and a reduction of neuronal excitability by inhibition of the voltage-gated sodium current. FIG. 5 shows that the FFA decreases the availability of voltage-gated sodium channels of hippocampal pyramidal neurons by shifting their voltage-dependent inactivation. No significant effect was detected on the activation curve.

In some embodiments, the present invention provides pharmaceutical compositions and methods for treating and/or preventing epileptic neuronal activity comprising administering to a subject a composition comprising FFA. The methods are not limited to a particular mechanism for treating and/or preventing chronic pain in a subject. In some embodiments, the administered FFA treats and/or prevents epileptic neuronal activity through activation of background potassium conductance. In some embodiments, the administered FFA treats and/or prevents epileptic neuronal activity through inhibiting voltage-gated sodium current. The methods are not limited to administering a particular amount of FFA. In some embodiments, the amount of administered FFA is sufficient to activate background potassium conductance and/or inhibit voltage-gated sodium current. In some embodiments, the amount of administered FFA is sufficient to reduce epileptic neuronal activity for the subject. In some embodiments, additional therapeutic agents are co-administered with the compositions comprising FFA. The methods are not limited to particular additional therapeutic agents. In some embodiments, the additional therapeutic agents include, but are not limited to, carbamazepine, clobazam, clonazepam, ethosuximide, felbamate, fosphenyloin, flurazepam, gabapentin, lamotrigine, levetiracetam, oxcarbazepine, mephenyloin, phenobarbital, phenyloin, pregabalin, primidone, sodium valproate, tiagabine, topiramate, valproate semisodium, valproic acid, vigabatrin, diazepam, lorazepam, paraldehyde, pentobarbital, and bromides.

The pharmaceutical compositions of the present invention (e.g., pharmaceutical compositions comprising FFA) may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

The present invention provides kits and pharmaceutical preparations for the treatment of conditions associated with neuronal dysfunction (e.g., chronic pain, epileptic neuronal activity). In some embodiments, the kits contain pharmaceutical compositions comprising FFA. In some embodiments, the kits contain pharmaceutical compositions comprising FFA and/or additional therapeutic agents.

In some embodiments, compositions of the present invention (e.g. FFA) are administered in any suitable dosage. In some embodiments, a subject is administered between 1 mg/kg (1 mg FFA per 1 kg body weight of the subject) and 100 mg/kg FFA (e.g. 1 mg/kg . . . 2 mg/kg . . . 5 mg/kg . . . 10 mg/kg . . . 20 mg/kg . . . 50 mg/kg . . . 100 mg/kg). In some embodiments, a subject is administered 1-20 mg/kg FFA (e.g. 1 mg/kg . . . 2 mg/kg . . . 3 mg/kg . . . 4 mg/kg . . . 5 mg/kg . . . 6 mg/kg . . . 7 mg/kg . . . 8 mg/kg . . . 9 mg/kg . . . 10 gm/kg . . . 11 mg/kg . . . 12 mg/kg . . . 13 mg/kg . . . 14 mg/kg . . . 15 mg/kg . . . 16 mg/kg . . . 17 mg/kg . . . 18 mg/kg . . . 19 mg/kg . . . 20 gm/kg). In some embodiments, a subject is administered 1-10 mg/kg FFA. In some embodiments, a subject is administered 10-50 mg/kg FFA. In some embodiments, a subject is administered between 1 and 2000 mg FFA (e.g. 1 mg . . . 2 mg . . . 5 mg . . . 10 mg . . . 20 mg . . . 50 mg . . . 100 mg . . . 200 mg . . . 500 mg . . . 1000 mg . . . 2000 mg). In some embodiments, a subject is administered 50-1000 mg FFA. In some embodiments, a subject is administered 100-800 mg FFA.

### EXPERIMENTAL

### Example 1

### Compositions and Methods

Hippocampal brain slices: 11-to 20-day-old Long-Evans rats were anesthetized with isoflurane and killed by decapitation. The brain was removed from the skull in ice-cold artificial cerebrospinal fluid (ACSF), containing: 125 mM NaCl, 25 mM NaHCO₃, 2.5 mM KCl, 1.25 mM NaH₂PO₄, 1.8 mM CaCl₂, 1 mM MgCl₂ and 2 mM 5 glucose, bubbled with 95% O₂ and 5% CO₂ (pH 7.4). Transverse hippocampal slices (300 μm thick) were cut using a vibroslicer (Dosaka) and stored in a solution containing: 87 mM NaCl, 25 mM NaHCO₃, 2.5 mM KCl, 1.25 mM NaH₂PO₄, 0.5 mM CaCl₂, 7 mM MgCl₂, 75 mM sucrose and 25 mM glucose, bubbled with 95% O₂ and 5% CO₂; slices were kept at 35° C. for 15-20 minutes and subsequently at 24-25° C.

Electrophysiological recordings: Slices were visualized with an Axioskop 2FS (Zeiss) upright microscope with a water-immersion 60× objective (0.9 NA, Olympus). For whole cell current-clamp recordings, the bath solution contained kynurenic acid (2 mM) and picrotoxin (0.1 mM) to block fast synaptic transmission, and pipettes were filled with internal solution consisting of: 140K-gluconate, 8 mM NaCl, 2 mM MgCl₂, 1 mM EGTA, 2 mM Na₂ATP 2, 0.1 mM NaGTP, 10 mM HEPES, pH 7.3 with KOH.

To study the gating properties of the sodium channels, voltage-clamp recordings were performed in the nucleated patch configuration, which allows almost ideal voltage clamp, even for fast sodium currents (28). Recordings were performed at 24-25° C. using an Axopatch 200B (Axon Instruments) patch-clamp amplifier. Data were sampled at 20 kHz and filtered at 10 kHz. Patch pipettes had resistance of 2.5-4 MΩ (in working solution) and were pulled from Corning #0010 lead glass (WPI) or borosilicate 0.4 mm-walled glass (Dagan). The internal solution was CsCl-based and contained: 140 mM CsCl, 10 mM NaCl, 2 mM MgCl₂, 10 mM EGTA, 2 mM Na₂ATP, 0.1 mM NaGTP, 10 mM HEPES 10, pH 7.3 with CsOH.

Capacitive transients were reduced by wrapping the pipettes in parafilm and compensated using the fast compensation circuitry of the amplifier. The whole-cell configuration was obtained and the pyramidal identity of cells was quickly confirmed by presence of a sag in the membrane potential in response to a hyperpolarizing current injection. The pipette was then slowly withdrawn while applying a constant negative pressure (−90 to −130 mbar). After excision, patches were held at −90 mV with a small pressure (−15 to −20 mbar). Sodium currents were then obtained by offline digital subtraction of traces in TTX from control traces.

Drugs were applied to the patches by using a multi-barrel system consisting of 4 glass capillaries (1.5 mm ID) glued together and connected to a syringe pump (WPIL). The patch-pipette carrying the nucleated patch was inserted into each of the pipes, each containing a different extracellular solution. The system ensures that the solution speed is the same in each barrel. For these recordings, the extracellular solution consisted of HEPES-buffered ACSF: 138 mM NaCl, 10 mM HEPES, 2.5 mM KCl, 2 mM CaCl₂, 1 mM MgCl₂ and 25 mM glucose (pH 7.3), 50 μM CdCl₂, mM TEA, and the drugs of interest.

All drugs were from Sigma, except TTX (Alomone). FFA was prepared as stock solution (200 mM in DMSO) and stored at 2-4° C. Working solutions were prepared daily. Control solutions contained DMSO at the same concentration as in the test solution. 4-Aminopyridine (4-AP) was prepared as stock solution (100 mM in HEPES-buffered ACSF) and stored at −30° C. TTX was prepared as stock solution (1 mM in H₂O) and stored at 2-4° C.

Data Acquisition and Analysis: data were transferred a Digidata 1322A (Axon Instruments) interface and acquired using P-Clamp 9 (Axon Instruments) software.

The voltage-dependence of activation was fit with a Boltzmann function f(V)=1/(1+exp(−(V−V_{1/2})/k)) raised to the third power, where V_{1/2} is the voltage at which the activation is half-maximal, and k is the slope factor. The voltage-dependence of inactivation was similarly determined using a Boltzmann function, f(V)=1/(1+exp(−(V−V_{1/2})/k)). The time course of recovery from inactivation was fit with a bi-exponential equation of the form, f(t)=1−(A1 exp(−t/τ1) +A2 exp (−t/τ2)), yielding two time constants (τ) and relative contribution (A).

Computational modeling: Simulations were performed on a dual core 1.83 Ghz Inspiron 640 PC using the fully implicit backward Euler integration method with a time step of 0.01 ms in the NEURON simulation environment (http://www.neuron.yale.edu/neuron/, version 5.8, (7, 18)). A modified version of a previously published six-state sodium current model was used (4). In the modified version, a slow inactivated state has been added in order to account for the presence of a slow phase in the recovery from inactivation and for the differences in the voltage dependence of the steady state inactivation measured using 50 ms and 200 ms long pre-pulses. The experimental data show that FFA had only minor effect on this slow inactivation process and, accordingly, the transition rates to the slow inactivated state are not affected by FFA in the model. In addition, in the modified model the voltage dependence of the activation rates was tuned to match the more depolarized half-activation voltage and a shallower voltage dependence of activation. The transition rates between different states were described by a general equation which helps to relate the transition rates to the changes in the Gibbs free energy W and the moving particle charge (17); α/β=1/(A+(1/r)*exp[−(W−z_{α/β}*v)/(kT/qₑ)].

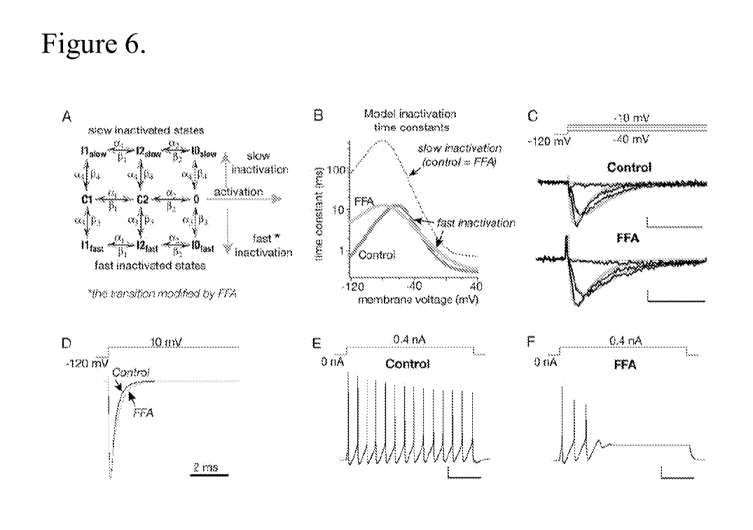

Here, α and βcorrespond to forward and backward transition rates as shown in FIG. 6; A accounts for the rate saturation; 1/r corresponds to the inverted Kramers escape rate, W is the Gibbs energy associated with the voltage independent conformational change; z_{α/β} is the product of the voltage sensor charge and the fraction of the membrane voltage that the particle senses during gating movements; k is the Boltzmann constant; T is the temperature and qₑ is the electron charge. The values of these parameters are provided in Table 1. It was assumed that FFA only affects the transition to the fast-inactivated state by modifying the corresponding z_{α/β} and W values. Moreover, it was assumed that the ratio of gating charges for this transition, Z_{α}/Z_{β}, was not affected by FFA. Thus, only two parameters were used and sufficient to simulate all FFA effects.

TABLE 1
Rate constants

| | q11 | A | 1/r | W (mV) | Z_{α,β} |
|---|---|---|---|---|---|
| α1 | 2.8 | 0 | 0.13 | −36.3 | 0.57 |
| β1 | 2.8 | 0 | 0.13 | −36.3 | 3.2 |
| α2 | 2.8 | 0.036 | 0.58 | −67.2 | 1.28 |
| β2 | 2.8 | 0.043 | 0.058 | −67.2 | 1.28 |
| α3 | 2.4 | 0.3 | 10 | −108.5 | 2.2 |
| β3 | 2.4 | 0 | 10 | −108.5 | 1.47 |
| α3 in FFA | 2.4 | 0.3 | 10 | −87.5 | 1.4 |
| β3 in FFA | 2.4 | 0 | 10 | −87.5 | 0.93 |
| α4 | 2.4 | 0.3 | 0.5 | −108.5 | 2.2 |
| β4 | 2.4 | 0 | 0.5 | −108.5 | 1.47 |

The general equation for rate constants is:
α, β = q11\hat{ }([23 − T]/10)/(A + (1/r) * exp[−, +(W − Z_{α} * V)/(kT/qe)] Here kT is equal to 25.6 mV for 24° C.

Voltage clamp recordings were simulated assuming [Na⁺]_{I}=5 mM and [Na⁺]_{O}=140 mM.

### Example 2

### Flufenamic Acid Decreases Neuronal Excitability and Modulates Voltage-Gated Sodium Channel Gating

Voltage-gated sodium currents were recorded in nucleated patches from CA I pyramidal cells from 11-to 20-day-old rats. FFA was tested at 0.2 mM, a concentration similar to that used as Ican blocker (0.5 mM, (32) and as two-pore potassium channels opener (0.1 mM, (40)).

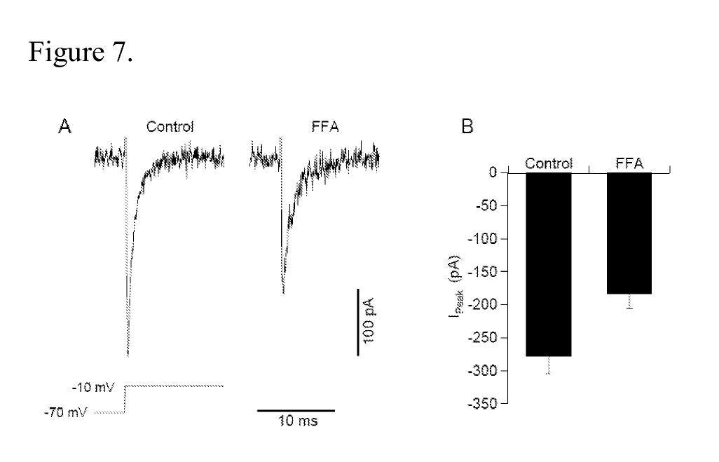

The effect of FFA on the amplitude of sodium currents recorded in nucleated patches was investigated using a 30 ms step (to 10 mV) from −70 mV (close to the resting potential of hippocampal pyramidal neurons). 200 μM FFA blocked 34% of the sodium current (FIG. 7). The mechanism of action of FFA on sodium channels was characterized in detail. Even with a cesium-based intracellular solution and in the presence of extracellular TEA and cadmium some contaminating currents could often still be detected in pyramidal cell nucleated patches; therefore analysis was performed on TTX-subtracted traces only. TTX-sensitive current data for the control and the FFA were obtained from different patches.

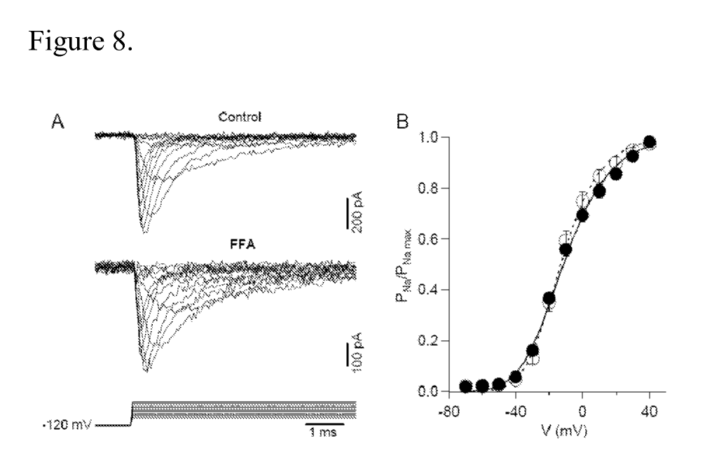

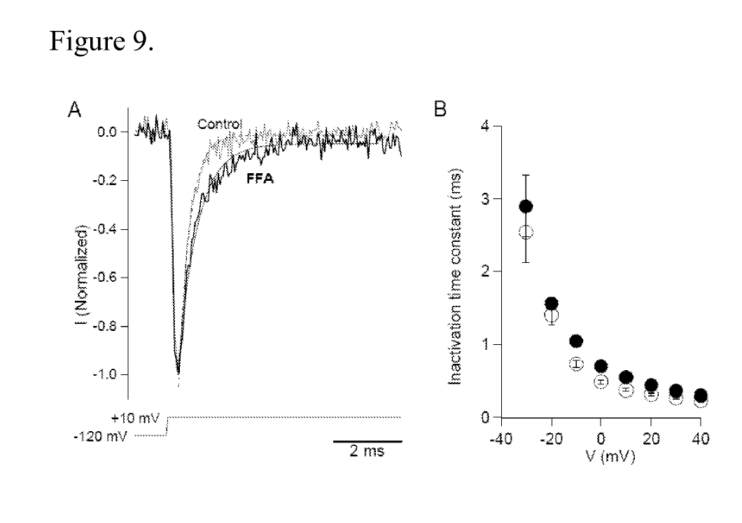

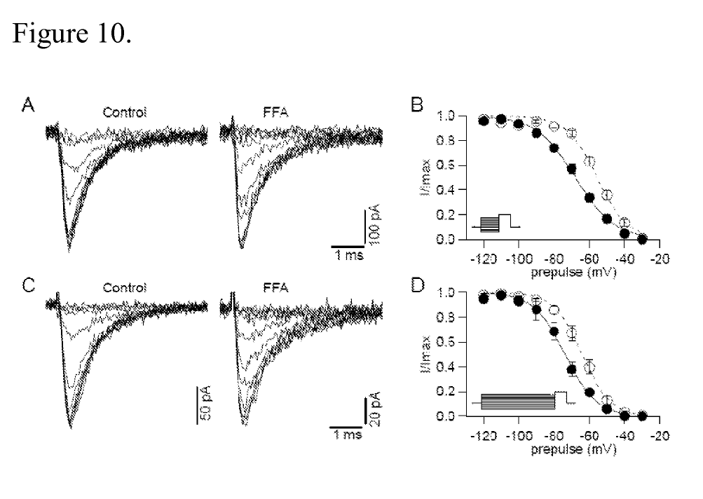

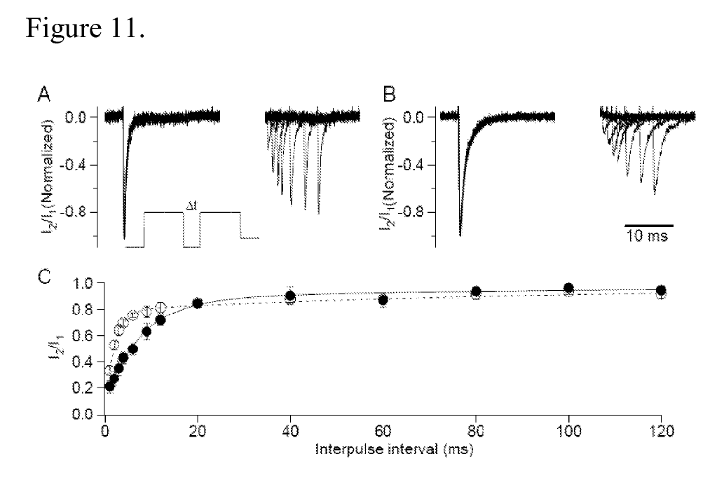

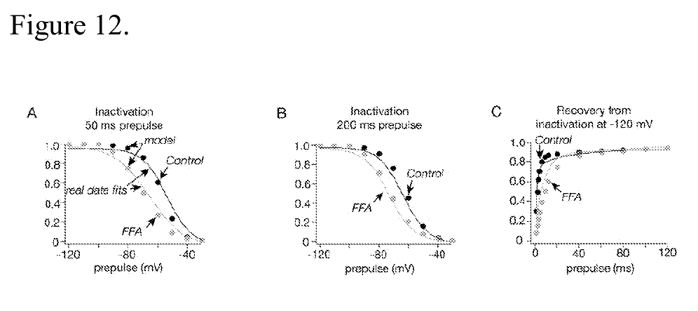

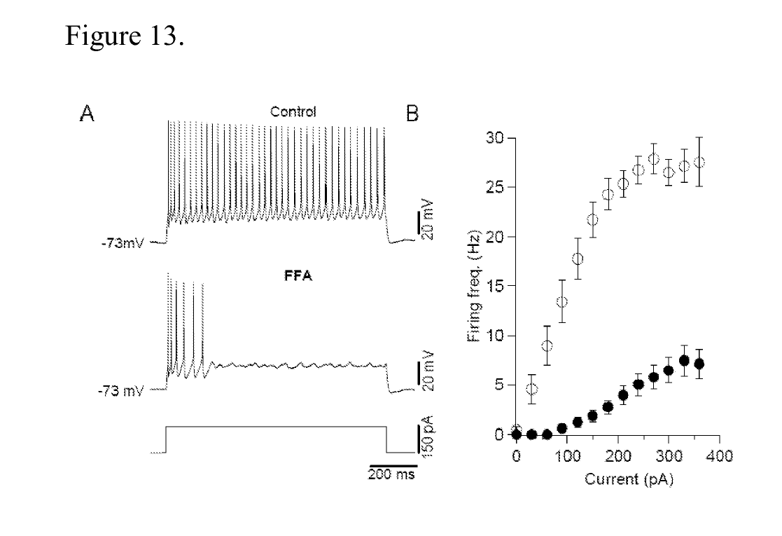

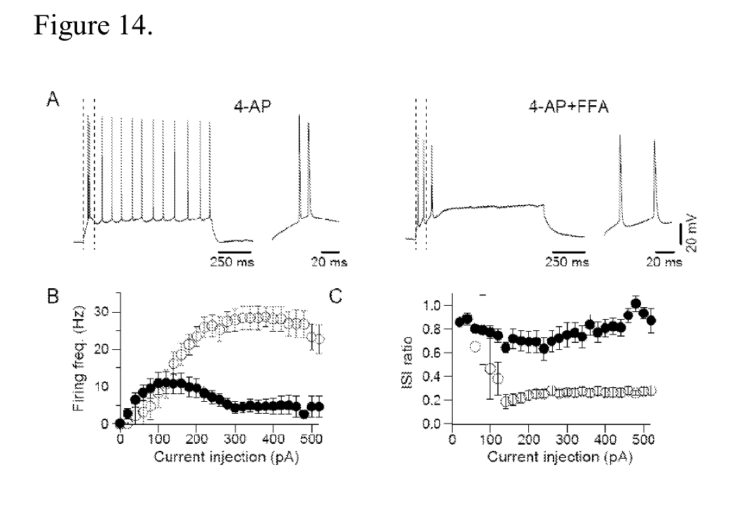

FFA had only minimal effect on the Na⁺ channel activation curve in hippocampal pyramidal cells (FIG. 8). Permeability/voltage plots showed that the activation curve was slightly affected by FFA (midpoint and slope factor were −12.5±1.78 mV and 4.5±0.25 mV in control versus −11.3±1.36 mV and 5.6±0.27 mV in FFA, respectively, FIG. 8B). In contrast, the inactivation process was strongly affected by FFA. The inactivation onset was fit with a single exponential function; in the presence of FFA, the onset of inactivation was significantly slower than in the control condition at most membrane potentials (FIG. 9; at 0 mV, the time constant was 0.48±0.03 ms in control and 0.70±0.03 ms in FFA. Thus, the mode of action of FFA appears to differ from typical use-dependent blockers such as carbamazepine or imipramine, which induce faster onset of inactivation (45). The effect of FFA on the voltage-dependent channel availability was analyzed. Patches were held at −90 mV, and tested using either a 50 ms-long or a 200 ms-long prepulse from −120 to −30 mV (10 mV steps) preceding a 30 ms-long test pulse to −10 mV (FIG. 10, insets). FFA dramatically shifted the steady-state inactivation curve to more hyperpolarized potentials; this shift explains the large reduction of the current amplitude at resting membrane potentials. The shift was similar for relatively short (50 ms, FIG. 10A,B) and for longer prepulses (200 ms, FIG. 10C,D). For 50 ms prepulse, the mid-point potential shifted from −55.4±1.3 mV in control, to −67.8±1.4 mV in FFA, and the slope factor from 8.3±0.4 to 10.7±0.5 mV. For 200 ms prepulse, the mid-point potential shifted from −64.1±2.2 mV in control, to −74.1±2.6 mV in FFA and the slope factor from 7.5±0.2 to 8.9±1.0 mV.

A large shift in the inactivation curve indicates that differences also exist in the process of the recovery from inactivation. A standard double pulse protocol, in which two identical voltage pulses (from −120 to −10 mV, 30 ms) are separated by an interval of increasing duration (FIG. 11 A,B), was used to study the de-inactivation process. Experiments conducted during development of embodiments of the present invention focused on the recovery in the millisecond range (12). The terms “Fast” and “slow” inactivation provide distinction between the two inactivated states characterized by faster and slower recovery, although even the slower recovery occurs with a time constant of <100 ms. Experiments conducted during development of embodiments of the present invention demonstrated that FFA reduced the speed of the de-inactivation process. As described previously (28), recovery from inactivation of the sodium channels of CA1 pyramidal neurons was best described by a bi-exponential function and this was the case in the presence of FFA as well (FIG. 11C). The fast and slow time constants were differentially affected by FFA; while the fast time constant was largely increased (−370%, from 2.1 ms in control to 7.7 ms in FFA) no appreciable effect was detected on the slow component (85 ms in control versus 61 in FFA, FIG. 11C).

The voltage-clamp data demonstrate that FFA strongly modulates Na⁺ channel availability and recovery from inactivation, which inhibit repetitive and burst firing. The effect of FFA on burst and tonic firing of hippocampal pyramidal neurons was examined during development of embodiments of the present invention (FIG. 13). Recordings were obtained in the presence of kynurenic acid (2 mM) and picrotoxin (0.1 mM) to block fast synaptic transmission. Under these conditions, FFA (0.2 mM) strongly decreased the number of action potentials elicited by a 1s-long depolarizing current injection. For a 150 pA depolarizing current pulse the firing frequency decreased from 21.7, 1.82 Hz in control conditions to 1.87, 0.61 Hz in FFA (FIG. 13A,B).

Experiments were conducted during development of embodiments of the present invention to examine the inhibitory effect of FFA on burst firing. All CA1 pyramidal cells show robust burst firing in the presence of 4-Aminopyridine (4-AP), a potassium channel blocker. Whole-cell current clamp recordings were obtained from CA1 pyramidal neurons in 4-AP bathed slices. These recordings were also performed at 32-33° C. and in the presence of kynurenic acid and picrotoxin. As expected, 4-AP induced robust burst firing (FIG. 14, black traces); bath application of FFA (0.2 mM) dramatically reduced both the total number of spikes and bursting (FIG. 14B,C), as quantified using the interspike interval ratio, which is inversely related to bursting (29, 30). The interspike interval ratio was significantly increased for all current injections larger than 120 pA (for 120 pA, the ISI ratio went from 0.38±0.14 in control to 0.74±0.06 in FFA, FIG. 14C).

Experiments conducted during development of embodiments of the present invention using a computational model to examine the mechanism of action of FFA. A modified version of a previously published model was used (4). The model was tuned to match the properties of sodium currents in hippocampal neurons such as a slightly different voltage dependence of activation and the presence of a slow phase in the recovery from inactivation (FIG. 6A, C, Table 1, Example 1). A slow inactivated state was added to account for the slow phase in the recovery from inactivation (FIG. 6A). FFA effect on sodium channels is mostly limited to the fast inactivation (FIG. 7-11). Accordingly, in the model the transitions to the fast inactivated state are the only ones affected by FFA. These transitions are described in the framework of the absolute rate theory that relates the transition rates to the Gibbs energy associated with the voltage independent conformational change during the gating and to the z factor, the product of the gating particle charge and the fraction of the membrane potential sensed by that particle. The forward and backward transitions between the open and fast inactivated states have different z factors that reflect different voltage dependence of inactivation and recovery from inactivation rates. It was assumed that FFA only modifies the sum Z_{α}+Z_{β}, but not the ratio Z_{α}/z. In addition, in our model FFA affected the Gibbs energy, W, associated with the voltage independent conformational change. Thus, all FFA effects were reproduced by a change in two model parameter values that modified the voltage dependence of the fast inactivation time constants (FIG. 6B). FIG. 6C shows that the simulated currents are similar to the ones recorded in hippocampal neurons and that FFA changed very little the activation properties of these currents while the inactivation rate was slower in FFA (FIG. 6D). Nevertheless, there was a dramatic change in excitability (FIG. 6E,F); although in control conditions a cell fired regular action potentials in, response to current injection (FIG. 6E), in the presence of FFA only few action potentials were fired and then the neuron became quiescent. These effects are explained by the reduced availability of sodium channels at membrane potentials close to the rest (FIG. 12), although the present invention is not limited to any particular mechanism of action and an understanding of the mechanism of action is not necessary to practice the present invention. In the model, FFA shifted inactivation voltage dependence to more hyperpolarized potentials (FIGS. 12A and B, filled circles), closely matching the experimental data (FIGS. 12A and B, thick lines). The recovery from inactivation at −120 mV was slowed down (FIG. 12C), also reproducing the experimental data. Thus, the proposed model accounts for all FFA effects on I Na⁺ and showed that sodium channel modulation can by itself fully account for the observed current clamp data, which result from the fact that during repetitive firing sodium channels accumulate into the inactivated state with each successive action potential leading to cessation of firing due to reduced sodium channel availability.

Mechanism of Na⁺ channel modulation by flufenamic acid: The shift in voltage dependence of inactivation (˜12 mV for a 50 ms prepulse) leads to a substantial reduction in the fraction of sodium channels available for activation at membrane potentials close to the resting in pyramidal neurons (from 0.86±0.02 in control to 0.57±0.04 in FFA at −70 mV, FIG. 7). However, because of the large safety factor for action potential generation in pyramidal neurons (26) and because of the negligible effect of FFA on the activation curve of sodium channels, single action potentials were little changed by FFA (FIG. 13). Repetitive firing, however, was largely reduced and burst firing abolished. This effect is reminiscent of the use-dependent block by lidocaine that has been attributed to a particularly high affinity of lidocaine to the inactivated channel (5, 16), and also is similar to the effects of numerous antiepileptic drugs such as phenyloin and carbamazepine (34). However, the mechanism of FFA action differs from the use-dependent block reported in the literature because the onset of inactivation becomes slower rather than faster and, crucially, there is no change in the slow phase of recovery from inactivation. In hippocampal pyramidal neurons the recovery from inactivation is best described by the sum of two exponential functions that represent the fast and the slow phase of recovery from inactivation (28). Use dependent block dramatically increases the slower phase of recovery from inactivation (23) that results into a more complete cumulative inactivation of sodium currents during application of brief repetitive depolarizing pulses (44). Such an increase in the slow cumulative inactivation is present if a drug causes a use-dependent inactivation (17, 34). Experiments conducted during development of embodiments of the present invention demonstrate no change in the slow phase of recovery from inactivation.

Simulations showed that FFA effects on sodium currents could be reproduced by assuming a change in both the on and off transition rates between the open and the fast inactivated state. To reproduce FFA effects, simulations the gating particle charge had to be reduced by ˜30% while the change in voltage independent conformational energy was <kT, here kT is the product of the Boltzmann constant, k, and the temperature, T. Since, in ideal gases, the average thermal energy of an atom is 1.5 times kT, a reduction in Gibbs energy of less than kT can be considered as marginal. Thus, FFA effects on sodium currents can be interpreted as screening by FFA of the fast inactivation gate voltage sensor.

Flufenamic acid abolishes burst firing: Under physiological conditions, about 15% of pyramidal neurons in the hippocampal CA1 area are burst firing (29). This firing phenotype is regulated by numerous conductances, some of which act synergistically and other antagonistically to determine the afterdepolarization that leads to burst firing. Voltage-gated sodium, calcium and potassium channels all play critical roles in shaping the afterdepolarization that leads to burst firing (3, 29, 30). Experiments conducted during development of embodiments of the present invention demonstrate that FFA effectively decreases repetitive firing in neurons depolarized with long-duration (1s) current injections (FIG. 13). Importantly, FFA completely abolishes burst firing in physiological conditions as well as in the 4-AP slice epilepsy model (2, 35) in which all CA1 pyramidal neurons are burst firing (FIG. 14). These effects depend on the sodium channel modulation, as reproduced in computational simulations in which the INa⁺ is the only current affected.

### REFERENCES

1. Aman, T. K., T. M. Grieco-Calub, C. Chen, R. Rusconi, E. A. Slat, L. L. Isom, and I. M. Raman. 2009. Regulation of persistent Na current by interactions between beta subunits of voltage-gated Na channels. J. Neurosci. 29: 2027-2042 PM:19228957.
2. Avoli, M., P. Perreault, A. Olivier, and J. G. Villemure. 1988. 4-Aminopyridine induces a long-lasting depolarizing GABA-ergic potential in human neocortical and hippocampal neurons maintained in vitro. Neurosci. Lett. 94: 327-332 PM:2849735.
3. Azouz, R., M. S. Jensen, and Y. Yaari. 1996. Ionic basis of spike after-depolarization and burst generation in adult rat hippocampal CA1 pyramidal cells. J. Physiol 492 (Pt 1): 211-223 PM:8730596.
4. Baranauskas, G., and M. Martina. 2006. Sodium currents activate without a Hodgkin-and-Huxley-type delay in central mammalian neurons. J. Neurosci. 26: 671-684 PM:16407565.
5. Bean, B. P., C. J. Cohen, and R. W. Tsien. 1983. Lidocaine block of cardiac sodium channels. J. Gen. Physiol 81: 613-642 PM:6306139.
6. Cantrell, A. R., J. Y. Ma, T. Scheuer, and W. A. Catterall. 1996. Muscarinic modulation of sodium current by activation of protein kinase C in rat hippocampal neurons. Neuron 16: 1019-1026 PM:8630240.
7. Carnevale, N. T., and M. L. Hines. 2006. The Neuron book. Cambridge University Press, Cambridge, UK.
8. Chao, T. I., and C. Alzheimer. 1995. Effects of phenyloin on the persistent Na+ current of mammalian CNS neurones. Neuroreport 6: 1778-1780 PM:8541480.
9. Chen, Y., F. H. Yu, E. M. Sharp, D. Beacham, T. Scheuer, and W. A. Catterall. 2008. Functional properties and differential neuromodulation of Na(v)1.6 channels. Mol. Cell. Neurosci. 38: 607-615 PM:18599309.
10. Coulter, D. A. 1997. Antiepileptic drug cellular mechanisms of action: where does lamotrigine fit in? J. Child Neurol. 12 Suppl 1: S2-S9 PM:9429123.
11. Fink, M., F. Duprat, F. Lesage, R. Reyes, G. Romey, C. Heurteaux, and M. Lazdunski.
1996. Cloning, functional expression and brain localization of a novel unconventional outward rectifier K+ channel. EMBO J.15: 6854-6862 PM:9003761.
12. Fleidervish, I. A., A. Friedman, and M. J. Gutnick. 1996. Slow inactivation of Na+ current and slow cumulative spike adaptation in mouse and guinea-pig neocortical neurones in slices. J. Physiol 493 (Pt 1): 83-97 PM:8735696.
13. Ghamari-Langroudi, M., and C. W. Bourque. 2002. Flufenamic acid blocks depolarizing afterpotentials and phasic firing in rat supraoptic neurones. J. Physiol 545:537-542 PM:12456832.
14. Grieco, T. M., J. D. Malhotra, C. Chen, L. L. Isom, and I. M. Raman. 2005. Open-channel block by the cytoplasmic tail of sodium channel beta4 as a mechanism for resurgent sodium current. Neuron 45: 233-244 PM:15664175.
15. Hervieu, G. J., J. E. Cluderay, C. W. Gray, P. J. Green, J. L. Ranson, A. D. Randall, and H. J. Meadows. 2001. Distribution and expression of TREK-1, a two-pore-domain potassium channel, in the adult rat CNS. Neuroscience 103: 899-919 PM:11301200.
16. Hille, B. 1977. Local anesthetics: hydrophilic and hydrophobic pathways for the drug-receptor reaction. J. Gen. Physiol 69: 497-515 PM:300786.
17. Hille, B. 2001. Ion channels of excitable membranes, third edition. Sinauer Associates, Sunderland, Mass.
18. Hines, M. 1993. NEURON—A program for simulation of nerve equations. In Neural systems: analysis and modeling. F. Eeckman, editor. Kluwer Academic Publishers, Norwell, Mass. 127-136.
19. Hodgkin, A. L., and A. F. Huxley. 1952. A quantitative description of membrane current and its application to conduction and excitation in nerve. 1952. Bull. Math. Biol. 52: 25-71 PM: 2185861.
20. Isom, L. L., K. S. De Jongh, D. E. Patton, B. F. Reber, J. Offord, H. Charbonneau, K. Walsh, A. L. Goldin, and W. A. Catterall. 1992. Primary structure and functional expression of the beta 1 subunit of the rat brain sodium channel. Science 256: 839-842 PM: 1375395.
21. Jung, H. Y., T. Mickus, and N. Spruston. 1997. Prolonged sodium channel inactivation contributes to dendritic action potential attenuation in hippocampal pyramidal neurons. J. Neurosci. 17: 6639-6646 PM: 9254676.
22. Kuo, C. C., R. S. Chen, L. Lu, and R. C. Chen. 1997. Carbamazepine inhibition of neuronal Na⁺ currents: quantitative distinction from phenyloin and possible therapeutic implications. Mol. Pharmacol. 51: 1077-1083 PM: 9187275.
23. Lang, D. G., C. M. Wang, and B. R. Cooper. 1993. Lamotrigine, phenyloin and carbamazepine interactions on the sodium current present in N4TG1 mouse neuroblastoma cells. J. Pharmacol. Exp. Ther. 266: 829-835 PM: 8394919.
24. Lee, A., and A. L. Goldin. 2008. Role of the amino and carboxy termini in isoform-specific sodium channel variation. J. Physiol 586: 3917-3926 PM: 18565993.
25. Ma, J. Y., M. Li, W. A. Catterall, and T. Scheuer. 1994. Modulation of brain Na⁺ channels by a G-protein-coupled pathway. Proc. Natl. Acad. Sci. U.S. A 91: 12351-12355 PM: 7991631.
26. Madeja, M. 2000. Do neurons have a reserve of sodium channels for the generation of action potentials? A study on acutely isolated CA1 neurons from the guinea-pig hippocampus. Eur. J. Neurosci. 12: 1-7 PM: 10651854.
27. Makita, N., P. B. Bennett, and A. L. George, Jr. 1996. Molecular determinants of beta 1 subunit-induced gating modulation in voltage-dependent Na⁺ channels. J. Neurosci. 16: 7117-7127 PM: 8929421.
28. Martina, M., and P. Jonas. 1997. Functional differences in Na+ channel gating between fast-spiking interneurones and principal neurones of rat hippocampus. J. Physiol 505 (Pt 3): 593-603 PM: 9457638.
29. Metz, A. E., T. Jarsky, M. Martina, and N. Spruston. 2005. R-type calcium channels contribute to afterdepolarization and bursting in hippocampal CA1 pyramidal neurons. J. Neurosci. 25: 5763-5773 PM: 15958743.
30. Metz, A. E., N. Spruston, and M. Martina. 2007. Dendritic D-type potassium currents inhibit the spike afterdepolarization in rat hippocampal CA1 pyramidal neurons. J. Physiol 581: 175-187 PM: 17317746.
31. Pace, R. W., D. D. Mackay, J. L. Feldman, and C. A. Del Negro. 2007. Inspiratory bursts in the preBotzinger complex depend on a calcium-activated non-specific cation current linked to glutamate receptors in neonatal mice. J. Physiol 582: 113-125 PM: 17446214.
32. Peña, F., M. A. Parkis, A. K. Tryba, and J. M. Ramirez. 2004. Differential contribution of pacemaker properties to the generation of respiratory rhythms during normoxia and hypoxia. Neuron 43: 105-117 PM: 15233921.
33. Raman, I. M., and B. P. Bean. 1997. Resurgent sodium current and action potential formation in dissociated cerebellar Purkinje neurons. J. Neurosci. 17: 4517-4526 PM: 9169512.
34. Rogawski, M. A., and W. Loscher. 2004. The neurobiology of antiepileptic drugs. Nat. Rev. Neurosci. 5: 553-564 PM: 15208697.
35. Rutecki, P. A., F. J. Lebeda, and D. Johnston. 1987. 4-Aminopyridine produces epileptiform activity in hippocampus and enhances synaptic excitation and inhibition. J. Neurophysiol. 57: 1911-1924 PM: 3037040.
36. Schiller, Y. 2004. Activation of a calcium-activated cation current during epileptiform discharges and its possible role in sustaining seizure-like events in neocortical slices. J. Neurophysiol. 92: 862-872 PM: 15277598.
37. Shaw, T., R. J. Lee, and L. D. Partridge. 1995. Action of diphenylamine carboxylate derivatives, a family of non-steroidal anti-inflammatory drugs, on [Ca2+]i and Ca(2+)− activated channels in neurons. Neurosci. Lett. 190: 121-124 PM: 7644119.
38. Srinivas, M., and D. C. Spray. 2003. Closure of gap junction channels by arylaminobenzoates. Mol. Pharmacol. 63: 1389-1397 PM: 12761350.
39. Swensen, A. M., and B. P. Bean. 2003. Ionic mechanisms of burst firing in dissociated Purkinje neurons. J. Neurosci. 23: 9650-9663 PM: 14573545.
40. Takahira, M., M. Sakurai, N. Sakurada, and K. Sugiyama. 2005. Fenamates and diltiazem modulate lipid-sensitive mechano-gated 2P domain K(+) channels. Pflugers Arch. 451: 474-478 PM: 16075240.
41. Tasso, S. M., L. E. Bruno-Blanch, and G. L. Estiu. 2001. Pharmacophore model for antiepileptic drugs acting on sodium channels. J. Mol. Model. 7: 231-239.
42. Taverna, S., T. Tkatch, A. E. Metz, and M. Martina. 2005. Differential expression of TASK channels between horizontal interneurons and pyramidal cells of rat hippocampus. J. Neurosci. 25: 9162-9170 PM: 16207875.
43. Wang, D., S. Grillner, and P. Wallen. 2006. Effects of flufenamic acid on fictive locomotion, plateau potentials, calcium channels and NMDA receptors in the lamprey spinal cord. Neuropharmacology 51: 1038-1046 PM: 16919683.
44. Willow, M., T. Gonoi, and W. A. Catterall. 1985. Voltage clamp analysis of the inhibitory actions of diphenylhydantoin and carbamazepine on voltage-sensitive sodium channels in neuroblastoma cells. Mol. Pharmacol. 27: 549-558 PM: 2581 124.
45. Yang, Y. C., and C. C. Kuo. 2002. Inhibition of Na(+) current by imipramine and related compounds: different binding kinetics as an inactivation stabilizer and as an open channel blocker. Mol. Pharmacol. 62: 1228-1237 PM: 12391287.

All publications and patents listed above and/or mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

We claim:

1. A method for treating a condition associated with neuronal dysfunction, comprising administering to a subject suffering from neuronal dysfunction a pharmaceutical composition comprising flufenamic acid.

2. The method of claim 1, wherein said condition associated with neuronal dysfunction is chronic pain.

3. The method of claim 2, wherein said pharmaceutical composition is co-administered with one or more therapeutic agents selected from the group consisting of antidepressants, selective serotonin reuptake inhibitors, opioids, anticonvulsants, analgesics, and nonsteroidal anti-inflammatory drugs.

4. The method of claim 1, wherein said condition associated with neuronal dysfunction is epileptic activity.

5. The method of claim 4, wherein said pharmaceutical composition is co-administered with one or more therapeutic agents selected from the group consisting of carbamazepine, clobazam, clonazepam, ethosuximide, felbamate, fosphenyloin, flurazepam, gabapentin, lamotrigine, levetiracetam, oxcarbazepine, mephenyloin, phenobarbital, phenyloin, pregabalin, primidone, sodium valproate, tiagabine, topiramate, valproate semisodium, valproic acid, vigabatrin, diazepam, lorazepam, paraldehyde, pentobarbital, and bromides.

6. The method of claim 1, wherein said FFA activates KCNK potassium channels.

7. The method of claim 1, wherein said FFA activates neuronal background potassium conductance.

8. The method of claim 1, wherein said FFA inhibits neuronal voltage-gated sodium current.

9. The method of claim 1, wherein said subject is a human.

10. A method for preventing a condition associated with neuronal dysfunction, comprising administering to a subject a pharmaceutical composition comprising flufenamic acid.

11. The method of claim 10, wherein said condition associated with neuronal dysfunction is chronic pain.

12. The method of claim 10, wherein said condition associated with neuronal dysfunction is epileptic activity.

13. The method of claim 10, wherein said FFA activates KCNK potassium channels.

14. The method of claim 10, wherein said FFA activates neuronal background potassium conductance.

15. The method of claim 10, wherein said FFA inhibits neuronal voltage-gated sodium current.
